(19) United States
(12) Patent Application Publication
(10) Pub. No.: US 2009/0099552 A1
(43) Pub. Date: Apr. 16, 2009

(54) Drug delivery route-based connector system and method

(76) Inventors: Maureen Levy, Tampa, FL (US); Donald Jones, Tampa, FL (US)

Correspondence Address: WALTER W. DUFT;LAW OFFICES OF WALTER W. DUFT, 8616 MAIN ST, SUITE 2, WILLIAMSVILLE, NY, 14221, US

(21) Appl. No.: 11/871,206

(22) Filed: Oct. 12, 2007

(51) Int. Cl. A61M 25/18

(52) U.S. Cl. 604/533

(57) ABSTRACT

A route-based connector for interconnecting a first drug delivery component with a second drug delivery component having a mating route-based connector. The connector has a geometrically-coded shape corresponding to a drug delivery route. A set of route-based connectors may be provided with plural geometrically-coded shapes that are each assigned to a corresponding drug delivery route according to a key. The connectors may also be color coded to designate the delivery route. A port adapter may be provided having a flow control valve for stopping and starting a flow of medication through the adapter. The port adapter may include a universal connector on one end thereof and a route-based connector on another end thereof that has a geometrically coded shape that allows the port adapter to connect to one of the route-based connectors. Machine readable or light emitting route identifiers may be provided on the connectors or on components associated therewith.

### BACKGROUND OF THE INVENTION

1. Field of the Invention

The invention relates generally to drug delivery systems. Still more particularly, the invention concerns connectors for interconnecting drug delivery components such as syringes, catheters, infusion bags, infusion pumps, tubing, intravenous ports, etc.

2. Description of the Prior Art

By way of background, there is presently no safeguard in the field of medicine against the practice of randomly interconnecting needle-less syringes, catheters, infusion bags, infusion pumps, tubing, intravenous ports and other drug delivery components. All such components use the same standard universal connectors (e.g., slip lock or luer lock tips) which makes them interchangeable and compatible with each other. This leads to increased medical risk due to the possibility of a medication designed for one delivery route being inadvertently delivered via another route. For example, if a syringe containing an intravenous medication is accidentally connected to an epidural port (or infusion bag) and the contents administered, the damage to the patient could be irreversible or fatal.

It is to improvements in the foregoing field that the present invention is directed. In particular, what is needed is an improved drug delivery technique whereby route delivery errors are minimized.

### SUMMARY OF THE INVENTION

The foregoing problems are solved and an advance in the art is obtained by a route-based connector for interconnecting a first drug delivery component with a second drug delivery component having a mating route-based connector. The connector has a geometrically-coded shape corresponding to a drug delivery route. A drug delivery system may be provided that includes a set of route-based connectors having plural geometrically-coded shapes that are each assigned to a corresponding drug delivery route. A key may be provided to define the delivery route assignments.

According to exemplary disclosed embodiments, the geometrically-coded shapes may include one or more of symmetric shapes or irregular non-symmetric shapes. The route-based connectors may also be color coded according to the delivery routes. The route-based connectors may include female connectors that each have an outer flange and an inner flange. One or both of the outer and inner flanges may have the geometrically-coded shape. The route-based connectors may further include male connectors having flanges that are adapted to be received into a gap between the outer and inner flanges of the female route-based connectors. The route-based connectors may be mounted on medication delivery components. The route-based connectors or the drug delivery components may comprise a machine-readable route identifier or a color-coded light emitter to help further verify the delivery route.

A port adapter may also be provided having a flow control valve for stopping and starting a flow of medication through the adapter. The port adapter may include a universal connector on one end thereof and a route-based connector on another end thereof having a geometrically coded shape that allows the adapter to connect to one of the route-based connectors.

### BRIEF DESCRIPTION OF THE DRAWINGS

The foregoing and other features and advantages of the invention will be apparent from the following more particular description of various exemplary embodiments, as illustrated in the accompanying Drawings, in which:

FIG. 1 is a partial perspective view of an exemplary route-based drug delivery system that includes a set of route-based connector pairs each having a female connector and a corresponding male connector;

FIG. 2 is a perspective view showing an exemplary chain of medication delivery components interconnected using route-based connector pairs that are geometrically-coded for a specific delivery route, and further illustrating the use of exemplary machine-readable route identifiers;

FIG. 3 is a perspective view showing an exemplary syringe having a color-coded light emitter on its plunger;

FIG. 4 is a front perspective view of an exemplary port adapter for adapting a route-based connector as shown in FIG. 1 to act as a universal connector;

FIG. 5 s an exploded front perspective view of the port adapter of FIG. 4;

FIG. 6 is an exploded rear perspective view of the port adapter of FIG. 4;

FIG. 7 is a front view of the port adapter of FIG. 4;

FIG. 8 is an exploded side view of the port adapter of FIG. 4;

FIG. 9 is a front perspective view of another exemplary port adapter for adapting a route-based connector as shown in FIG. 1 to act as a universal connector;

FIG. 10 is a rear perspective view of the port adapter of FIG. 9;

FIG. 11 is a perspective view of a syringe adapted to engage the port adapter of FIG. 9;

FIG. 12 is a view of the interior face of a female receptor providing a bayonet lock mechanism of the port adapter of FIG. 9;

FIG. 13 is a perspective view of the syringe of FIG. 11 engaging the port adapter of FIG. 9;

FIG. 14 is an internal cross-sectional view of the port adapter of FIG. 9 with a portion thereof broken away to illustrate internal flow control valve structure;

FIG. 15 is a perspective view of the port adapter of FIG. 9 with a portion thereof broken away to illustrate internal flow control valve structure; and

FIG. 16 is a perspective view of a main body of the port adapter of FIG. 9.

### DETAILED DESCRIPTION OF EXEMPLARY EMBODIMENTS

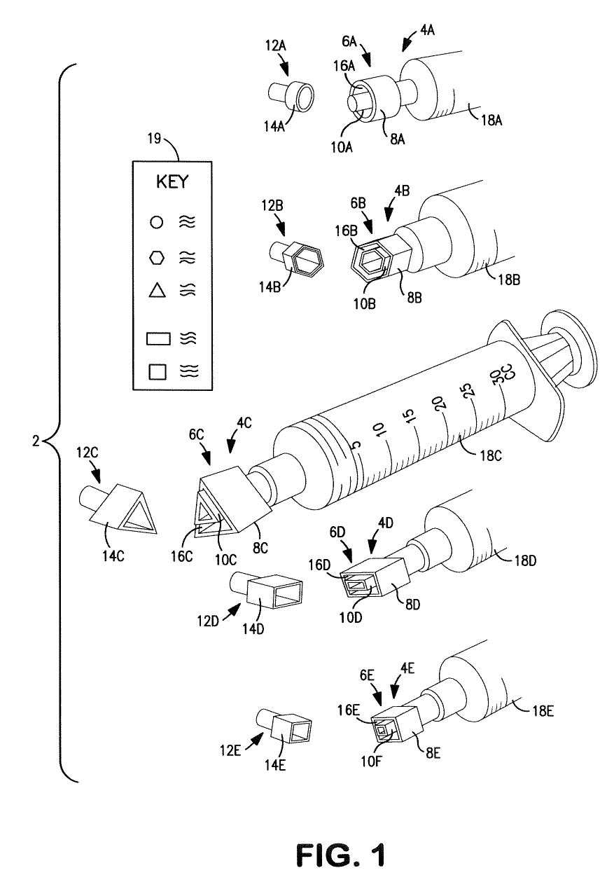

Turning now to the drawing figures, wherein like reference numerals represent like elements in all of the several views, FIG. 1 illustrates an exemplary route-based drug delivery system 2. The delivery system 2 is based on a set of exemplary route-based connector pairs 4A-4E, each of which is geometrically coded with a unique geometric shape that can be designated for a specific route of medication administration. Each route-based connector pair 4A-4E includes a respective female connector 6A-6E that includes a respective outer flange 8A-8E and a respective inner flange 10A-10E that defines a centrally disposed fluid delivery port for medication. Each route-based connector pair 4A-4E also includes a respective male connector 12A-12E having a respective flange 14A-14E that fits into a respective gap 16A-16E between the outer flange 8A-8E and the inner flange 10A-10E of the female connector 6A-6E.

The outer flanges 8A-6E and the inner flanges 10A-10E of the female connectors 6A-6E provide female connector portions that are geometrically-coded to respectively engage the mating male connector portions provided by the flanges 14A-14E of the male connectors 12A-12E. The connector portions of each connector pair 4A-4E of the drug delivery system 2 comprise the different geometrically-coded shapes that correspond to the different drug delivery routes to which the geometrically-coded shapes are assigned. These geometrically-coded shapes are selected so that only connectors having connector portions with matching geometrically-coded shapes may be interconnected to form a connector pair, and so that connectors having connector portions with non-matching geometrically-coded shapes may not be interconnected.

One or both of the outer flanges 8A-8E and the inner flanges 10A-10E of the female connectors 6A-6E may have the geometrically-coded shape. In FIG. 1, both the outer flanges 8A-8E and the inner flanges 10A-10E are geometrically-coded. For example, the female connector 6A has outer and inner flanges 8A/10A that are both of circular geometry, the female connector 6B has outer and inner flanges 8B/10B that are both of hexagonal geometry, the female connector 6C has outer and inner flanges 8C/10C that are both of triangular geometry, the female connector 6D has outer and inner flanges 8D/10D that are both of rectangular geometry, and the female connector 6E has outer and inner flanges 8E/10EB that are both of square geometry.

Other geometrically-coded shapes may also be used for the outer flanges 8A-8E and the inner flanges 10A-10E of the female connectors 6A-6E. Furthermore, the geometric coding could be based on the use of a single shape that is offered in different sizes, much like a socket wrench set provides a set of sockets of different size. Each size would then be assigned to a different delivery route. However, although the use of size-based geometric coding in this manner would be technically feasible, it would not allow medical personnel to visually distinguish connectors for different delivery routes as easily as if different connector shapes were used.

It would also be possible to fashion either the outer flanges 8A-8E or the inner flanges 10A-10E of the female connectors 6A-6E using a universal connector configuration (e.g., with a slip lock or luer lock tip), so long as the other flange is geometrically coded. If this is to be done, it is preferable to provide the outer flanges 8A-8E with the unique geometric shape in order to facilitate visual identification of the route-based connectors and their assigned route. According to this example, only the inner flanges 10A-10E of the female connectors 6A-6E would have a universal configuration. The inner surfaces of the male connector flanges 14A-14E would of course be appropriately modified to accommodate the universal shape of the female connector inner flanges 10A-10E. Although not shown in FIG. 1, the delivery system 2 may also include a universal connector for non-critical drug delivery routes such as intramuscular administration, topical use or for a combination of non-critical routes.

The female connectors 6A-6E are adapted to connect only to respective counterpart male connectors 12A-12E having geometrically compatible configurations assigned to the same delivery route. Neither the female connectors 6A-6E nor the male connectors 12A-12E are able to cross fit with connectors having other shapes for other delivery routes because their design will not allow them to do so. For each respective female connector 6A-6E there will be a corresponding respective male connector 12A-12E, and visa versa. As stated, each male connector 12A-12E that connects to a female connector 4A-4E will do so by virtue of its flange 14A-14E (providing the male connector portion) fitting into the gap 16A-16E between the outer flange 8A-8E and the inner flange 10A-10E of the female connector (providing the female connector portion). It will be seen that the flange 14A of the male connector 12A has a circular geometry, the flange 14B of the male connector 12B has a hexagonal geometry, the flange 14C of the male connector 12C has a triangular geometry, the flange 14D of the male connector 12D has a rectangular geometry, and the flange 14E of the male connector 12E has a square geometry.

The geometrically-coded shapes of the route-based connector pairs 4A-4E shown in FIG. 1 are defined relative to a viewing plane that is transverse to a connector axial direction. The connector axial direction will usually be the principal direction used for coupling and uncoupling the route-based connector pairs 4A-4E (i.e., the coupling direction). For example, manipulation of a female and male connector 6A-6E and 12A-12E toward each other in the connector axial direction will tend to couple the connectors together to form one of the route-based connector pairs 4A-4E. Conversely, movement of the assembled female and male connector 6A-6E and 12A-12E away from each other in the connector axial direction will tend to uncouple the connectors and disassemble the connector pair 4A-4E. The connector axial direction will typically also correspond to the medication flow pathway. In FIG. 1, the geometrically-coded shape of the connector portion of each route-based connector pair 4A-4E is the shape that is seen when looking toward the female and male connectors 6A-6E and 12A-12E in the connector axial direction. In this orientation, the geometrically-coded shapes will be perceived to surround a central connector longitudinal axis that defines the connector axial direction. In most cases, the geometrically-coded shape will be symmetrically formed around the connector longitudinal axis. However, this is not a requirement and irregular non-symmetrical geometrically-coded shapes could also be used, such as patterns with irregularly lobed or undulating side walls.

The non-connecting end of the female connectors 6A-6E may be mounted to any standard drug delivery route component, including not only the needle-less syringes 18A-18E shown in FIG. 1, but also catheters, infusion bags, infusion pumps, tubing, intravenous ports, other connectors, etc. The male connectors 12A-12E may be similarly mounted to a drug delivery component that needs to be attached to the drug delivery component that mounts a female connector 6A-6E. Thus, if the female connectors 6A-6E are mounted to the needle-less syringes 18A-18E, the male connectors 12A-12E could each be mounted to a patient drug delivery port, or to a tube extending to such a port, or to an infusion pump connected to such a tube, etc. To minimize the possibility of drug delivery errors, there will preferably be a route-based connector pair 4A-4E disposed at each connection interface between adjacent components in the drug delivery pathway. A chain of drug delivery components and route-based connector pairs 4A-4E will thus be created according to treatment requirements. Unlike conventional luer lock tips where there are no incompatible connections, the route-based connectors pairs 4A-4E are geometrically designed to be compatible only with all other connectors of the same route according to a geometric-coding/deliver route assignment key (see below). The female connectors 6A-6E and the male connectors 12A-12E cannot cross fit with connector shapes designated for other routes because the connector portions will not match.

The route-based connector pairs 4A-4E may also be color coded with a unique color code that can be designated for the delivery route associated with the route-based connector geometric shape. As a further visible aid in the process of differentiating routes, the same color coding used on the route-based connector pairs 4A-4E may be added to the delivery route components, such as the plungers of the syringes 18A-18E. Color codes could be similarly applied to catheters, infusion bags, infusion pumps, tubing, intravenous ports, and other components.

The route-based connector pairs 4A-4E thus provide a mechanical lockout system and may further include an optional visual verification system that helps medical practitioners identify medications and verify that their route of administration is correct. For example, a medication designated to be administered into the central nervous system via an epidural/spinal route can be identified for that route by assigning the route to one of the route-based connector pairs 4A-4E. The geometric shape of the selected route-based connector pair 4A-4E will be required for all connectors in the chain of drug delivery components from the medication source to the point of entry into the patient. This will prevent the central nervous system medication from being administered via any route other than the epidural/spinal route.

An example of this situation is a high concentration of a medication for intrathecal infusion placed in an implanted pump infusion. The aim is to prevent the medication from being given via an intravenous route. The route-based delivery system 2 provides mechanical confirmation that the correct route of administration has been selected and, if the route-based connector pairs 4A-4E are color coded, allows the medical practitioner to visually verify the delivery route through which the medication is to be administered.

In order to define and manage the assignment of geometrically-coded shapes (and colors) to specific delivery routes, the route-based delivery system 2 may further include a key 19 that provides a record of the route assignments. The key 19 could be implemented using any suitable technology that allows the route assignments to be established and maintained. Implementation options may range from something as simple as a slip of paper containing printed assignment information to devices that record the assignment information in machine readable form. Relative to printed keys, examples would include laminated cards, tags, charts, posters, labels, etc. The key 19 could also be a distributed key provided by printing or otherwise placing (e.g., molding) delivery route information directly on the connectors themselves or on the drug delivery components associated therewith. Relative to machine-readable keys, examples include RFID tags on the connectors or associated drug delivery components, portable (e.g., bedside) devices and database management systems.

An exemplary set of geometric codes and color assignments for various drug administration routes that could be recorded by the key 19 is presented in Table 1 below. These assignments are shown by way of example only, and it will be appreciated that any desired combination of geometry-color-route assignments could be made, depending on implementation preferences. The assignments may be established on a hospital-by-hospital basis, but more preferably will be adopted by a state or national medical association or other standards body for widespread standardized use.

TABLE 1

| Systems | Geometry | Color |
|---|---|---|
| Intravascular | Circle | Red |
| Intravenous | | |
| Arterial | | |
| Central Nervous | Hexagon | Blue |
| Spinal (intrathecal) | | |
| Central Nervous System | | |
| Ventricular System | | |
| Epidural/Central Nervous | Triangular | Yellow |
| Caudal Epidural Nervous | | |
| Peripheral Nervous | | |
| Oral/Nasal/GI Track | Rectangular | Green |
| Oral | | |
| Intranasal Rectal | | |
| Rectal | | |
| Internal Cavities/Visceral | Square | Gray |
| Intra peritoneal | | |
| Intra pleural | | |
| Intra visceral | | |
| Intra uterine | | |
| Integument | Universal | Orange |
| Muscular Skeletal | | |
| Intra-lesion | | |
| Subcutaneous | | |
| Intramuscular | | |
| Intra-articular | | |
| Topical | Universal | Purple |
| Combination System | Universal | Yellow |

The delivery system 2 may be used according to an exemplary route-based delivery method to administer medications with a high degree of confidence that delivery route errors will not be made. The method begins with the selection of drug delivery components that are needed to deliver a medication from a medication source to a point of entry into the patient. The components are selected according to the desired delivery route for the medication being administered and based on the route-based connector geometry code and/or color code that has been specified for the desired route by way of the key 19. For example, using a key implementing Table 1 above, if it is desired to deliver a medication intravenously, only delivery components having the circular female and male connectors 6A and 12A will be selected. If color is part of the selection criteria, then the female and male connectors 6A and 12A could all have red color coding (if Table 1 is used). Once all of the delivery components have been selected in this manner, their female and male connectors 6A and 12A can be interconnected together to form a delivery line beginning at a medication source (e.g., a needle-less syringe) and terminating at an intravenous port deployed intravenously into the patient. In this way, there is no possibility that a syringe containing a medication belonging to another delivery route can be administered. Its route-based connector would not match any of the route-based connector pairs 4A chosen for the intravenous route.

As a further check, a source of medication, such as a syringe, vial, bag, etc., may be tagged with a machine-readable route identifier, such as an RFID (Radio Frequency IDentification) tag, containing a delivery route code that identifies the correct delivery route for the medication. When the source component is attached to a downstream component using one of the route-based connector pairs 4A-4E, the medical practitioner may scan the machine-readable route identifier to verify that the medication is compatible with the delivery route. Additional scanner functionality may also be provided, such as recording information regarding the medication administration event, including the patient name or identification number, the date, the time, the practitioner who performed the administration, the medication, and the delivery route. Such information may be captured by the scanner and stored (e.g., uploaded to a database) for future reference (e.g., as a medical record). The information capture may be performed by scanning the machine-readable route identifier, and possibly also by scanning a patient chart or ID bracelet to obtain information not provided by the machine-readable identifier.

If desired, additional delivery route components, or the connector pairs 4A-4E themselves, may be tagged with a machine-readable route identifier containing a delivery route code. As stated above, this represents one way that the key 19 could be implemented. Whenever a female or male connector 6A-6E or 12A-12E on one component is attached to a corresponding connector on another component, the medical practitioner may scan a machine-readable route identifier on each side of the connection to verify the delivery route. This could act as a further safety check and provide a record of how the delivery route was setup. If desired, each component and/or female or male connector 6A-6E and 12A-12E in a delivery route could be tagged, and medical personnel could be required to perform a tag scan each time a component interconnection is made as the delivery route is set up.

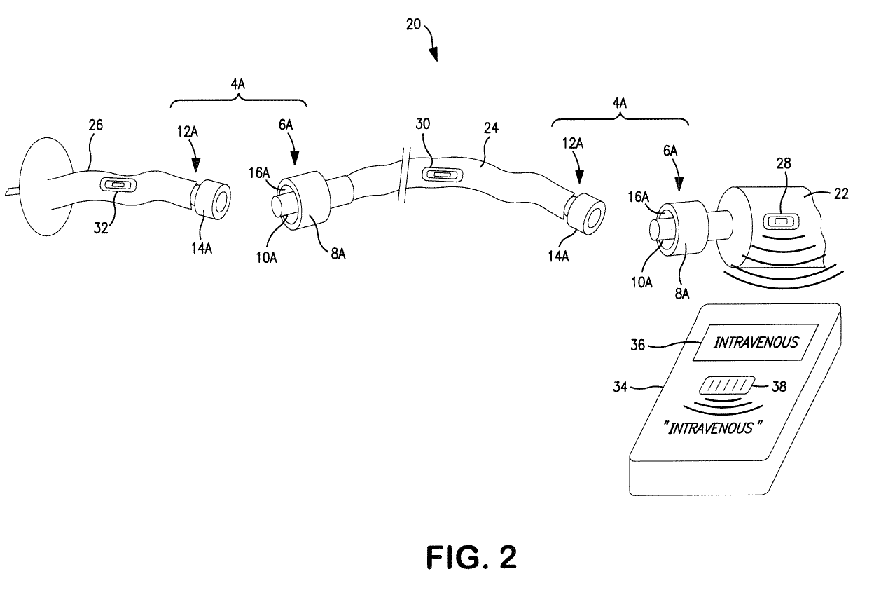

FIG. 2 illustrates an example of the foregoing technique in an exemplary chain 20 of intravenous delivery route components, namely, a syringe 22 containing an intravenous medication, tubing 24 connected to the syringe, and an intravenous port 26 connected to the tubing 24. The syringe 22 and the tubing 24 are interconnected by a first route-based connector pair 4A of FIG. 1, with the female connector 6A being on the syringe 22 and the male connector 12A being on one end of the tubing. The tubing 24 and the intravenous port 26 are interconnected by a second route-based connector pair 4A of FIG. 1, with the female connector 6A being on the other end of the tubing 24a and the male connector 12A being on the intravenous port. The syringe 22, the tubing 24 and the intravenous port 26 are respectively provided with machine-readable route identifiers 28, 30 and 32. As mentioned above, the machine-readable route identifiers 28, 30 and 32 could also be provided on the female and male connectors 6A and 12A as an alternative or in addition to providing them on the delivery components 22, 24 and 26. The route identifiers 28, 30 and 32 are each implemented as RFID tags. Other machine-readable technologies, such as bar codes, could also be used. An RFID scanner 34 has been placed in proximity to the route identifier 28 on the syringe 22 and is shown to be reading information therefrom. A visual display 36 on the scanner 34 verifies that the delivery route is the correct “INTRAVENOUS” route. The scanner may also include an audio output component 38, such as a speaker or buzzer, that may be used to provided an audible confirmation that a delivery component is compatible with the intended delivery route. To use this feature, the medical practitioner could specify the delivery route to the scanner 34 by performing an appropriate input operation. Then, when the scanner 34 reads one of the route identifiers, the audio output component 38 could either audibly verify whether the component is compatible with the delivery route. A compatibility indication could be generated as a specific tone (e.g., a pleasing sound) or by generating a synthetic voice output (e.g., of the word “intravenous”). An incompatibility indication could be generated as a specific tone (e.g. a series of harsh beeps) or as a synthetic voice output (e.g., of the word “incompatible” or of a word identifying the actual delivery route associated with the component).

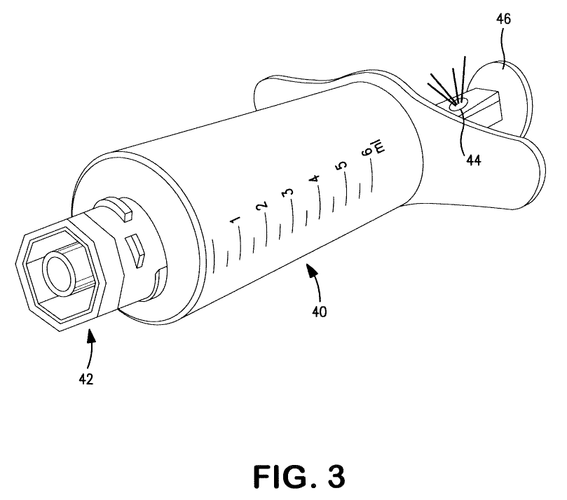

FIG. 3 illustrates a further alternative wherein a syringe 40 with a route-based connector 42 is provided with a color-coded light emitter 44 on its plunger 44. The light emitter 44 may be implemented in any suitable fashion. For example, it may be electrically implemented as an LED (Light-Emitting Diode) or other electrical device powered by a solar cell or small battery. It may also be chemically implemented by using a luminescent chemical device. A photoluminescent light emitter may also be used. The light emitter 44 could also be formed on other portions of the syringe 40, or on other delivery route components, or on the connector pairs 4A-4E themselves.

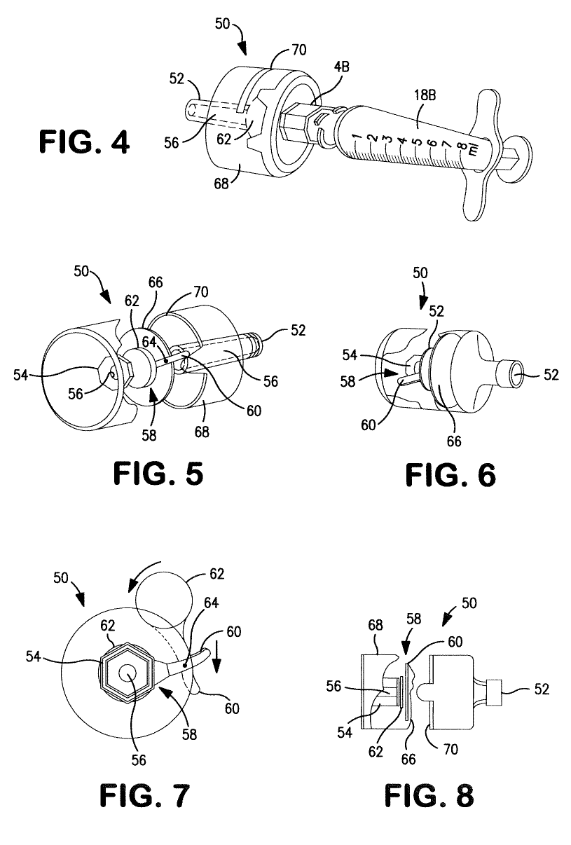

Turning now to FIGS. 4-8, the delivery system 2 may further include a universal port adapter 50 that allows any of the female or male connectors 6A-6E and 12A-12E to be used as a universal connector. The port adapter 50 also acts as a valve to prevent the flow of medication until it is verified that the medication is being administered via the correct route. The port adapter 50 includes a universal connector 52 at one end thereof. A male (or female) route-based connector 54 is provided at the other end. A fluid passage 56 extends through the port adapter 50 but can be closed by a flow-blocking valve 58 (see FIGS. 5-8) that can be opened and closed using a manually operable valve handle 60. Any suitable valve design may be used, including valves that are wholly or partially automated). FIGS. 4-8 show one possible construction in which the valve 58 includes a flow blocker 62 attached to the handle 60. The handle is pivotally mounted at 64 to an annular support plate 66 disposed within the port adapter's main body 68. The central opening of the annular support plate 66 forms part of the fluid passage 56. The flow blocker 62 may be formed as a relatively thin disk made of a suitable seal material that is moved into alignment with the fluid passage 56 when the valve is closed and moved out of alignment with the fluid passage when the valve is opened. As best shown in FIGS. 4, 5 and 8, the main body 68 is formed with a slot 70 to accommodate movement of the valve handle 60 and the flow blocker 62 during the valve opening and closing operations.

The port adapter 50 helps reduces the risk of medication delivery route error prior to administering a drug to the patient by preventing the medication from being released until all necessary queried are made. The valve 58 provides a protective barrier that forces the administrator of the medication to take conscious action by moving the handle 60 only after all other safeguards are met. The port adapter 50 is thus an additional safety protective device that may be utilized before a medication is administered to the patient. At the initial stage of the delivery process, a medication source (e.g., a needless syringe) having a female or male connector 6A-6E and 12A-12E may be connected to the port adapter 50, but the medication will be blocked by the valve 58 until proper checks have been made. One way that such a check could be performed is by scanning a machine-readable route identifier as described above. Note that the port adapter 50 may also be color coded to the route of administration associated with its route-based connector 54. The port adapter 50 could also be provided with a machine readable or light emitting identifier as described above. It will also be appreciated that the port adapter 50 need not have the universal connector 52 thereon, and could instead have a route-based connector at both ends.

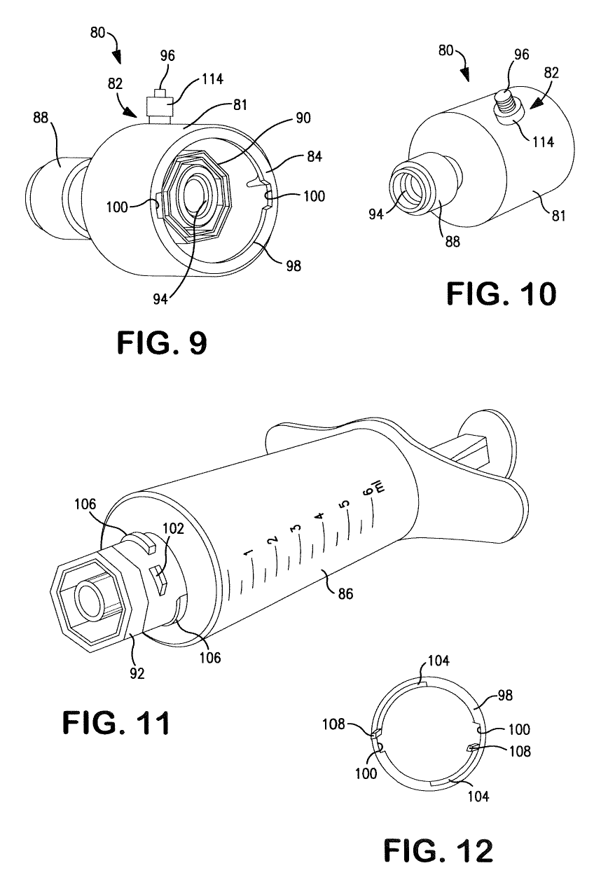

Turning now to FIGS. 9-11, an alternative universal port adapter 80 is shown having a modified flow control valve 82 and a lock mechanism 84 for locking the adapter to a delivery route component, such as a syringe 86. The port adapter 80 includes a main body 81 having a universal connector 88 at one end thereof. A male (or female) route-based connector 90 is provided at the other end that matches a corresponding route-based connector 92 on the syringe 86. A fluid passage 94 extends through the port adapter 80 but can be closed by the flow control valve 82, which includes a manually operable valve pushbutton 96. As additionally shown in FIG. 12, the lock mechanism 84 may be implemented using a bayonet mount female receptor 98 comprising a pair of slots 100 adapted to receive a pair of pins 102 on the syringe 86. A pair of arcuate ramps 104 on the interior side of the female receptor 98 engage the pins 102 as the syringe 86 is rotated, pulling the syringe 86 toward the port adapter 80 until a pair of circumferential ribs on 106 on the syringe engage the body of the receptor 98. A pair of tabs 108 on the interior side of the receptor 98 engage the pins 102 to stop the rotation of the syringe 86.

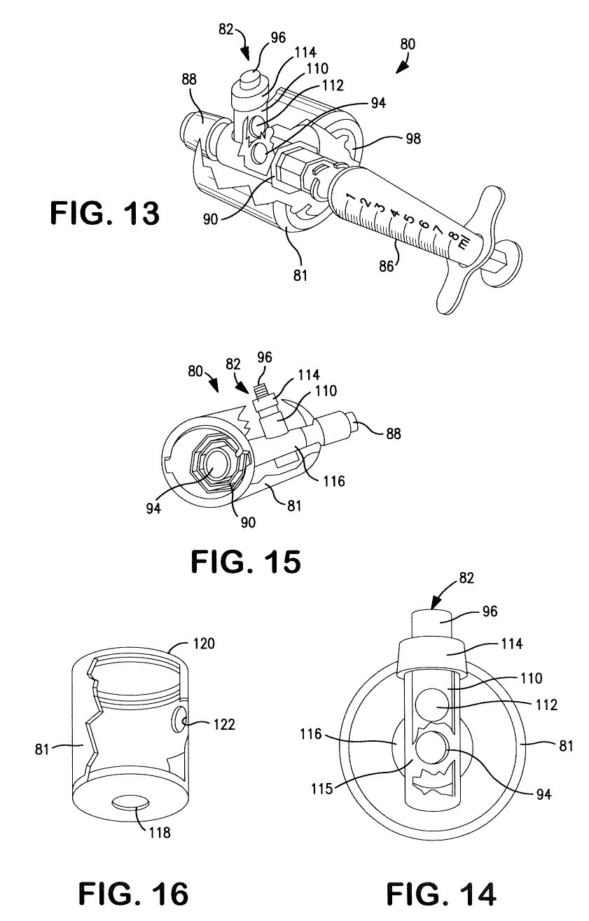

FIG. 13 shows the syringe 86 connected to the port adapter 80. It also shows an exemplary internal construction of the valve 82 in which a reciprocating valve element 110 has a fluid port 112 that aligns with the fluid passage 94 when the valve pushbutton 96 is depressed. When the pushbutton 96 is released, a biasing element (not shown) pushes the valve element 110 upwardly so that the fluid port 112 is no longer aligned with the fluid passage 94 and the flow of fluid through the port adapter 80 is blocked. This arrangement is additionally illustrated in FIG. 14. A rotatable ring member 114 is threadably mounted as part of a compression fitting that allows the pushbutton 96 to be locked in either the up (valve closed) or down (valve open) position. A blocking portion 115 of the valve element 110 blocks the fluid passage 94 when the valve 82 is in the closed position. As shown in FIG. 15, the valve element 110 is slideably carried in a main fitting 116 that defines the fluid passage 94. FIG. 16 shows the port adapter's main body 81. A small opening 118 at one end receives the end of the main fitting 116 that carries the universal connector 88. A large opening 120 at the other end carries the female receptor 98 of the lock mechanism 84. A side port 122 receives the valve element 110.

Like the port adapter 50, the port adapter 80 helps reduces the risk of medication error prior to administering a drug to the patient by preventing the medication from being released until all necessary queried are made. The valve 82 provides a protective barrier that forces the administrator of the medication to take conscious action by activating the pushbutton 96 only after all other safeguards are met. The port adapter 80 is thus an additional safety protective device that may be utilized before a medication is administered to the patient. At the initial stage of the delivery process, a medication source (e.g., a needless syringe) having a female or male connector 6A-6E and 12A-12E may be connected to the port adapter 80, but the medication will be blocked by the valve 82 until proper checks have been made. One way that such a check could be performed is by scanning a machine-readable route identifier as described above. As is the case with the port adapter 50, the port adapter 80 may also be color coded to the route of administration associated with its route-based connector 90. The port adapter 80 could also be provided with a machine readable or light emitting identifier as described above. It will also be appreciated that the port adapter 80 need not have the universal connector 88 thereon, and could instead have a route-based connector at both ends. Also, in lieu of a pushbutton operated control valve 82, the valve could be implemented as a rotatable petcock valve or using any other suitable valve design.

Accordingly, a route-based delivery system and related components have been disclosed, together with a method for administering a medication with delivery route verification being achieved using geometrically coded and/or color coded connectors, together with optional scanning of machine-readable route identifiers. While exemplary embodiments have been shown and described, it should be apparent that many variations and alternative embodiments could be implemented in accordance with the teachings herein. It is understood, therefore, that the invention is not to be in any way limited except in accordance with the spirit of the appended claims and their equivalents.

What is claimed is:

1. A drug delivery system, comprising:
a set of route-based connector pairs each having a female connector and a male connector with inter-engaging connector portions;
said connector portions of each connector pair having different geometrically-coded shapes that correspond to different drug delivery routes to which said geometrically-coded shapes are assigned;
said geometrically-coded shapes being selected so that only connectors having connector portions with matching geometrically-coded shapes may be interconnected, and so that connectors having connector portions with non-matching geometrically-coded shapes may not be interconnected; and
a key comprising a record of assignments of said geometrically-coded shapes to said drug delivery routes.

2. A drug delivery system in accordance with claim 1, wherein said geometrically-coded shapes include one or more of non-circular symmetric shapes or irregular non-symmetric shapes.

3. A drug delivery system in accordance with claim 1, wherein said route-based connector pairs are color coded according to said drug delivery routes.

4. A drug delivery system in accordance with claim 1, wherein said female connectors have an outer flange and an inner flange, and wherein one or both of said outer and inner flanges have said geometrically-coded shape.

5. A drug delivery system in accordance with claim 4, wherein said inner flange defines a central fluid delivery port.

6. A drug delivery system in accordance with claim 4, wherein said male connectors have a geometrically shaped flange adapted to be received into a gap between said outer and inner flanges of said female connectors.

7. A drug delivery system in accordance with claim 1, further including a port adapter having a flow control valve for stopping and starting a flow of medication through said port adapter.

8. A drug delivery system in accordance with claim 7, wherein said port adapter comprises a universal connector on one end thereof and a route-based connector on another end thereof that has a geometrically-coded shape that allows said port adapter to connect to one of said route-based connectors.

9. A drug delivery system in accordance with claim 1, wherein one or more of said route-based connector pairs are mounted on medication delivery components.

10. A drug delivery system in accordance with claim 9, wherein said route-based connector pairs or said medication delivery components comprise one or more of a machine-readable route identifier or a light emitter that is color coded according to one of said delivery routes.

11. A drug delivery method, comprising:
selecting two or more drug delivery components for administering a medication via a predetermined drug delivery route;
said drug delivery components having route-based connectors that are shaped according to a geometrically-coded shape associated with said predetermined drug delivery route by way of a key;
said drug delivery components being selected based upon said route-based connector geometrically-coded shape and using said key to exclude geometrically-coded shapes that do not correspond to said predetermined drug delivery route; and
interconnecting said drug delivery components using said route-based connectors in order to form said predetermined drug delivery route.

12. A drug delivery method in accordance with claim 11, wherein said geometrically-coded shape includes one or more of non-circular symmetric shapes or irregular non-symmetric shapes.

13. A drug delivery method in accordance with claim 11, wherein said route-based connectors are each color coded according to said predetermined drug delivery route, and wherein said drug delivery components are further selected based upon said route-based connector color coding.

14. A drug delivery method in accordance with claim 11, wherein said route-based connectors comprise female connectors having an outer flange and an inner flange, and wherein one or both of said outer and inner flanges have said geometrically-coded shape.

15. A drug delivery method in accordance with claim 14, wherein said inner flange defines a central fluid delivery port.

16. A drug delivery method in accordance with claim 14, wherein said route-based connectors further comprise male connectors having a geometrically shaped flange adapted to be received into a gap between said outer and inner flanges of said female connectors.

17. A drug delivery method in accordance with claim 11, further including selecting a port adapter having a flow control valve for stopping and starting a flow of medication through said port adapter.

18. A drug delivery method in accordance with claim 17, wherein said port adapter comprises a universal connector on one end thereof and a route-based connector on another end thereof that has a geometrically coded shape that allows said port adapter to connect to one of said route-based connectors.

19. A drug delivery method in accordance with claim 11, wherein said route-based connectors or said drug delivery components comprise a machine-readable route identifier and wherein said method further includes scanning said route identifier to verify that said drug delivery components are compatible with said predetermined drug delivery route.

20. A route-based connector for interconnecting a first drug delivery component with a second drug delivery component having a mating route-based connector, said route-based connector having a geometrically-coded shape transverse to a connector coupling direction that is adapted to slideably engage a matching geometrically-coded shape of said mating route-based connector, said geometrically-coded shape corresponding to a drug delivery route and being selected from either a shape group consisting of non-circular symmetric shapes, or a shape group consisting of irregular non-symmetric shapes.

21. A route-based connector in accordance with claim 20, wherein said connector is color coded according to said drug delivery route.

22. A route-based connector in accordance with claim 20, wherein said connector comprises a female connector having an outer flange and an inner flange, and wherein one or both of said outer and inner flanges have said geometrically-coded shape.

23. A route-based connector in accordance with claim 22, wherein said inner flange defines a central fluid delivery port.

24. A route-based connector in accordance with claim 20, wherein said connector further comprises a male connector having a geometrically shaped flange adapted to be received into a gap between an outer and inner flange of a corresponding female route-based connector.

25. A route-based connector in accordance with claim 20, wherein said connector forms part of said first drug-delivery component.
